(19) United States
(12) Patent Application Publication
(10) Pub. No.: US 2026/0102590 A1
(43) Pub. Date: Apr. 16, 2026

(54) Catheter Securement Device

(71) Applicant: Paul Alan Thomas, Schertz, TX (US)

(72) Inventor: Paul Alan Thomas, Schertz, TX (US)

(73) Assignee: Secure Connections, LLC, Schertz, TX (US)

(21) Appl. No.: 18/917,544

(22) Filed: Oct. 16, 2024

(51) Int. Cl. A61M 25/02

(52) U.S. Cl. CPC A61M 25/02

(57) ABSTRACT

Pertaining to an improved catheter securement device for securing medical implements such as IV devices to the body of patients. The device comprises a base, a platform, and a securement member, where the bottom surface of the base has an adhesive for adhering the device to a patient. Mounted atop the base is the angled platform and securement member. The securement member has a method of latching to close around IV devices or tubing and acts as a physical barrier to movement. The platform and securement member are of a rigid material, while the base is somewhat flexible.

### BACKGROUND OF THE INVENTION

It is necessary to administer medications and fluids directly into the veins of patients through IVDs. These IVDs undergo various pulling forces during routine use, therefore it is imperative the IVDs are properly secured such that these pulling forces do not cause the accidental removal or malpositioning of the device. Securement is currently achieved through different means from sutures to adhesive tape, and a limited number currently available CSDs.

Any IVD movement can lead to complications. The goal previously has been primarily to secure or fix the IVD to the patient's skin. Because of this, previous designs do not add much direct support or strain relief to the device or tubing. As a result, pulling forces often compromise the sterile dressing, which is installed to protect the sterility of the IV site. A compromised dressing must be replaced, in a sterile manner requiring significant time.

Some IVDs, are uniquely shaped and there are no currently available CSDs that may be utilized to secure them, therefore they must be sutured into place. According to groups like the CDC, OSHA, and the Infusion Nurse's Society, sutures are no longer recommended for securing IVDs because of an increased risk of infection. Additionally, I have observed that uniquely shaped IVDs, like sheath introducers, are problematic to secure as they require part of the device tubing to be repositioned in order to appropriately apply the necessary transparent film dressing. The dressing is then applied on top of this repositioned tube resulting in a lifting force being generated under the dressing, which often peels the dressing off the skin. Thus, the dressing is compromised. This may allow potentially harmful bacteria to enter the bloodstream, requiring at a minimum, the removal and replacement of the IVD and treatment with strong antibiotics.

For other IVDs, such as PICC lines and central lines, there are some previously available CSDs. However, these devices can be overly complex with excessive parts. Some have multiple latches which often require the use of both hands for proper installation. They use delicately small and easily breakable parts. Some previously available CSDs employ and adhesive which fixes the IVD to itself and requires the IVD to be pulled to separate. Many of these features become problematic and may result in the IVD being unintentionally pulled out. Any movement or pulling out of the IVD is a complication which will result in the necessary removal and replacement of the IVD. I have observed many doctors not using CSDs but instead continuing to use sutures or staples to secure the central lines they insert despite current recommendations not to.

Peripheral IVs are typically only secured by tape which has disadvantages. There are many different techniques for applying tape, all of which are time consuming and require preparation. Prior to IV insertion, tape is typically torn into multiple thinner strips and temporarily placed on a nearby surface during the insertion. I have observed that once the IV is in place, the tape is transferred to the clean IV site from the nearby surface. This surface was never disinfected, because cleaning nearby surfaces is not a typical step in the IV insertion process. The tape is potentially carrying any number of germs to the clean IV site. Additionally, there is no decidedly best technique for applying the tape for securement, so I have encountered varying degrees of effectiveness of these techniques. Poorly secured IVs lead to frequent IVD changes and increased needle sticks for patients.

Other problems with previous designs are that IVs are inserted at an angle through the skin and into the vessel but are routinely bent in order to lie flat on the skin. If the IVD is lying flat on the skin, the tubing must also bend as it passes through the skin because the angle of insertion doesn't change through the skin and vessel. I have often observed this bending of the tubing which impedes the flow and function of the IVD. Nurses refer to this type of IV being “positional”. Positional IVs have a shorter duration of use and must be replaced frequently.

Another drawback of securing a device directly to the skin is that the hard plastic of the device puts pressure on the skin causing skin breakdown. I have observed patients who suffer skin breakdown after an IV is taped directly to the skin. These patients are at an increased risk of infection due to the compromised skin and must undergo a new and painful IVD to be inserted.

An improved CSD maximizes the life of the IV by limiting IVD movement. Not using sutures or potentially dirty tape can decrease infection potential. A better CSD will also decrease precious time spent securing the IVD and also prevent interruptions in therapy by maintaining maximum flow and function of the IV and simplifying nurse's jobs. Thus, an improved CSD that replaces the need for sutures or complicated taping techniques and is simpler and easier to use with fewer parts. This simpler design aims to reduce possible complications for patients while also saving time for nurses and clinical staff and therefore improving patient care.

My CSD is advantageous over previous designs in that it:
Was created and designed by nurses with practical use in mind
Is simple and user friendly using fewer parts
Takes less time to apply than current CSDs
Replaces the need for tape
Mitigates device movement
Maintains the angle of entry reducing kinking and improving flow
Provides a layer of protection between the device and skin
Provides support and strain relief directly to the IVD

One embodiment is advantageous when using uniquely shaped devices like sheath introducers in that it:
Eliminates suturing decreasing infection potential
Avoids having to reposition IVD tubing
Maximizes dressing integrity

One embodiment is advantageous over previously available CSDs in that it:
Utilizes walls to limit device movement
Utilizes a retractable wall to avoid repositioning the IVD

### SUMMARY OF THE INVENTION

I, Paul Thomas of Schertz, TX have created an improved, simple and unique Catheter Securement Device (CSD). CSDs are necessary to secure and limit the movement and prevent the accidental removal of IV Devices (IVD). My CSD is comprised of a base layer, a platform, and a securement member and may be used on the majority of IVDs

### BRIEF DESCRIPTION OF DRAWINGS

FIG. 1 shows one embodiment of this CSD from a partially rotated frontal perspective view in the closed position

FIG. 2 shows the same device from FIG. 1 in a partially rotated perspective view in the closed position with a sheath introducer IVD in place.

FIG. 3 shows the same device of FIG. 1 from a frontal perspective in the open position.

FIG. 4 shows an alternative embodiment of this CSD from a partially rotated frontal perspective view in the open position with a PICC line within and retractable wall lowered.

FIG. 5 shows the same device of FIG. 4 in the closed position with a PICC line secured within.

FIG. 6 shows an alternative embodiment of this CSD from a partially rotated frontal perspective view.

FIG. 7 shows the same device of FIG. 6 from an opposite view with a peripheral IV secured within.

### DETAILED DESCRIPTION OF THE INVENTION

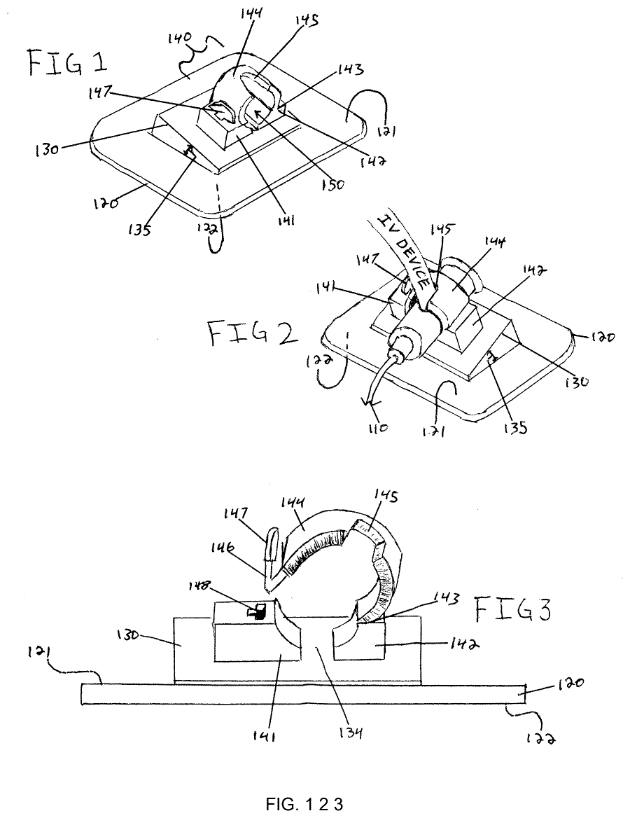

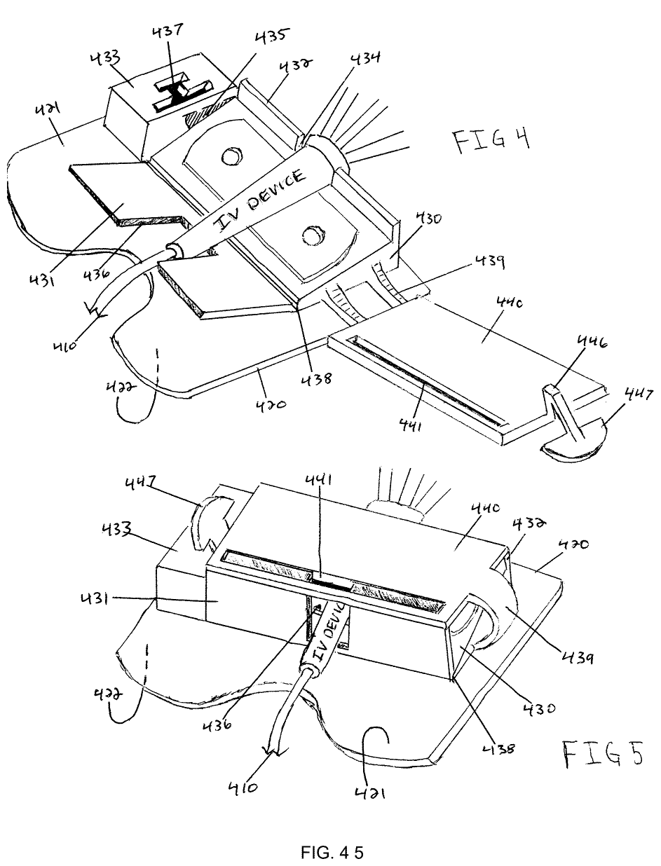

One embodiment of this CSD as seen in FIGS. 1-3 is comprised of a base layer 120, a platform 130, and a securement member 140. The base 120 has a non-adhesive upper side 121 and an opposite adhesive underside 122 for securing the device to the body. Attached to the upper side 121 is an angled platform 130 upon which sits a securement member 140. Platform 130 has about it an angle of decline 135 which maintains the angle at which the IVD was inserted. Securement member 140 is connected to platform 130 through a receiver base 141 and an opposite second base 142. Lying between bases 141 and 142 is a cutout or channel 134 which allows a device's suture loop to rest within.

Extending from base 142 is a cincture arm 144 which is connected to base 142 by a flexible hinge-like connection 143. Arm 144 is further comprised of a “U” shaped channel 145 within which allows medical tubing to pass through. On arm 144, opposite hinge 143 is a latching mechanism 146. Latch 146 has a terminal end latch tab 147. This latch 146 can be inserted into latch receiver 148 which is housed within latch base 141.

When latch 146 is inserted into receiver 148 a circular ring-like space 150 is created. This space 150 is where the IVD body will be secured with the side port extension tubing passing through channel 145, as seen in FIG. 2. Channel 145 provides strain relief and direct support to an IVD and/or tubing and allows the device and tubing to not be repositioned.

At the time of removal or a dressing change latch tab 147 is depressed which will allow latch 146 to slide out of receiver 148 and arm 144 may be moved away from the IVD body. The entire CSD can be removed from under the IVD and away from the insertion site. A new CSD can be slid into place under the IVD, arm 144 then approximated and latch for proper securement.

The combined structure of the base pad 120, the platform 130, along with the structures of securement member 140 provide for securing IVDs such a sheath introducers to the body without them having to be sutured in place. Additionally, the combined structure of this device is advantageous over sutures in that it directly supports and stabilizes the IVD and tubing while also mitigating the movement and rotation of the IVD in multiple direction planes. Furthermore, my CSD maintains the desired angle of entry and places a layer of protection between the IVD and the skin.

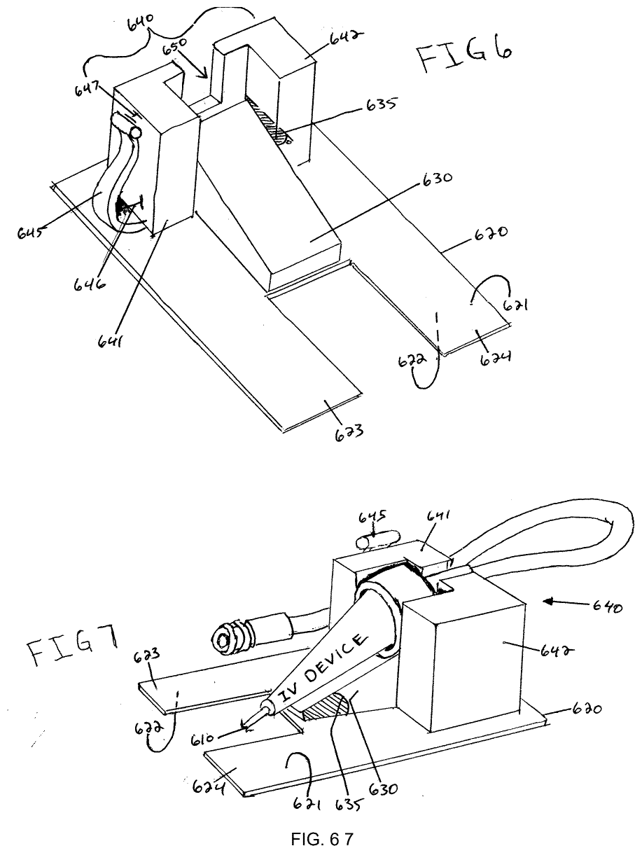

An alternative embodiment of this CSD as seen in FIGS. 4 and 5 is comprised of a base layer 420, a platform 430, and a securement member 440. The base 420 has a non-adhesive upper side 421 and an opposite adhesive underside 422 for securing the device to the body. Fixed to the upper side 421 is an angled platform 430 upon which an IVD will rest. Extending from one side of platform 430 is a fixed wall 432 and from an opposite side, a retractable wall 431. Additionally, platform 430 has on one side a receiver base 433, which contains a receiver 437. Platform 430 connects to a securement member 440 through a hinge-like connection 439. Platform 430 has an angle of decline 435 which maintains the angle at which the IVD was inserted. Extending up from the taller side of platform 430 the fixed wall 432 which acts as a barrier to movement, and it further has a window-like cutout 434 through which the IVD and/or tubing will pass and exit the device. Opposite wall 432 is retractable wall 431, also a barrier to movement, which joins to platform 430 at the lower height of angle 435 via a hinge-like structure 438 allowing wall 431 to be lowered or retracted in order to avoid IVD repositioning. Contained within wall 431 is a window-like cutout 436 through which the IVD tubing will enter into the device. Windows 434 and 436 provide strain relief from routine pulling forces.

Securement member 440 acts as the top or clamping member of this device. Securement member 440 contains a slot 441 through which the tope edge of wall 431 will protrude when in the “closed” position and allow wall 431 to become locked in place. Securement member 440 has an end opposite hinge 439 where a latching mechanism 446 is located. Latch 446 contains a latch tab 447. When hinge 439 is flexed and securement member 440 is positioned on top of platform 430, latch 446 will slide into receiver 437 locking it into a “closed” position. The rectangular cubic space created from closing the securement member 440 onto platform 430, and between walls 431 and 432, and between receiver base 433 and hinge 439 is where an IVD will rest as depicted in FIG. 5.

During a routine dressing change or at the time of removal, latch tab 447 is pressed, allowing latch 446 to be released from receiver 437. Securement member 440 is then able to open up and move away from platform 430 exposing the IVD. Wall 431 is then able to be lowered down out of the way of the IVD as depicted in FIG. 4. The entire CSD is then able to be slid out from under the IVD rather than the IVD having to be repositioned. A new sterile CSD may be slid into place under the IVD wall 431 folded up. And member 440 latched into place securing and supporting the IVD.

The combined structure of the base 420, the securement member 440, along with the structures of platform 430 provide for securing IV devices, such as PICC lines and Central lines to the body using fewer delicate parts, and through a simpler design that can be opened and closed with one hand. The angle 435 maintains the angle of insertion improving flow and function. The retractable wall eliminates the need for the IVD to be raised up off of the CSD during a dressing change. This lowers the risk of accidentally pulling the line out which requires a specialist to insert a new IVD at a significant financial cost and a significant amount of time. Additionally, my CSD provides direct support and stabilization to IVDS and tubing while also mitigating movement and rotation of the IVD in multiple directional planes.

An alternative embodiment of my CSD as seen in FIGS. 6 and 7 is comprised of a base pad 620, a platform 630, and a securement member 640. The base 620 has a non-adhesive upper side 621 and an opposite adhesive underside 622 for securing the device to the body. Base 620 contains at least two extensions 623 and 624 which extend toward the point where an IV device enters the skin 610. The combination of base 620 and extensions 623 and 624 act to provide stability and support to the device and prevent the pulling out of the IVD.

Fixed upon the upper side 621 is a securement member 640 having a first side 641 and an opposite second side 642 extending up. Sides 641 and 642 have a central channel 650 between where medical tubing and the connection to the IVD rest. Sides 641 and 642 act to minimize lateral movement and provide strain relief from pulling forces on medical tubing. Attaching to at least one side of securement member 640 is a means of latching 645 which secures and holds a loop or end of medical tubing. Latch 645 is comprised of a somewhat flexible yet sturdy material and containing an opening 646 between the body of latch 645 and securement member 640 which has a similar size to the diameter of medical tubing where which said tubing will rest. This tubing will enter into latch 645 via an opening 647. Tension created by securing medical tubing within latch 645 will additionally help to hold the IVD securely within the securement member 640.

Additionally fixed upon base upper side 621 and extending from securement member 640 is an angled platform 630 upon which the IVD will rest, as seen in FIG. 7. Platform 630 has about it an angle of decline 635 which maintains the angle at which the IVD was inserted, and additionally acts as a protective layer between the IVD and a patient's skin. A transparent film dressing (which is not part of this application but is a key component used in current practice, and which is standard in IV start kits) will help hold the IVD securely to the CSD.

The combined structure of the base pad 620, the structures of platform 630, along with the structures of securement member 640 provide for securing IVDs such as peripheral IVs to the body without the use of tape. Additionally, the combined structure of this device provides is advantageous over tape in that it directly supports and stabilizes the IVD and tubing while also mitigating the movement and rotation of the IVD in multiple directional planes. Furthermore, my CSD maintains the desired angle of entry and places a layer of protection between the IVD and the skin.

1. A securement device for IV devices and catheters comprising:
a base pad having an upper side and an opposite underside where at least a portion of the undersurface having an adhesive thereon for securing the device to the body; and
a platform attached to the upper side of the base pad having an angle of decline about its upper surface upon which a securement member will rest;
said securement member comprising a cincture arm of predetermined length and a first and second base extending up where the first base having a receiver therein and the second base having a means of connection and articulation with said cincture arm;
where said cincture arm is further comprising an elongation having a cutout or notch therein and a terminal end having a means of latching to and from the securement receiving base, whereby the approximation and latching of the cincture arm to the base creates an annulus of predetermined size where an IV device will securely rest;
whereby the combined structure of the base pad, the platform, along with the bases, cincture arm and latch of the securement member provide for securing IV devices to the body and additionally providing direct support and stabilization to IV devices and tubing while also mitigating the movement and rotation of the IV device in multiple planes.

2. The securement device of claim 1 where the base pad further comprising a solid yet generally flexible material such as but not limited to fabric, foam, or other synthetic materials.

3. The securement device of claim 1 where the base comprising the use of any medical-grade adhesive safe for us on skin and further comprising at least one set of release paper attached to the bottom surface of the base as a means of temporarily covering the adhesive until the device may be optimally positioned.

4. The securement device of claim 1 where the platform is generally rectangular and where the angle of decline about the platform is between 10 and 45 degrees.

5. The securement device of claim 1 where the platform, and securement member are comprised of a solid material such as but not limited to any injection-moldable plastics.

6. The securement device of claim 1 where the means of connection between the securement base and the cincture arm comprising but not limited to any living hinge or other mechanism allowing flexion.

7. The securement device of claim 1 where the means of latching is comprised of any latching mechanism that may be closed and therefore locked by simple insertion of the latch into the receiver and which may be opened or unlocked by depressing a tab connected to a lever-like appendage on the latch.

8. A securement device for IV devices and catheters comprising:
a base pad having an upper side and an opposite underside where at least a portion of the undersurface having an adhesive thereon for securing the device to the body; and
a platform attached to the upper side of the base pad having an angle of decline about its upper surface as a means of maintaining an angle of IV device insertion and upon which an IV device will rest;
said platform further comprising a fixed wall extending from one side and a retractable wall extending from an opposite side, and having on one side a receiving base and an opposite side having a connection to a securement member;
a securement member comprising an elongation with one end having a means of connection to and articulation with the platform and an opposite end having a means of latching the securement member to and from the platform base;
whereby the combined structure of the base pad, the securement member, along with the base, walls, and receiver of the platform provide for securing IV devices to the body and additionally providing direct support and stabilization to IV devices and tubing while also mitigating movement and rotation of the IV device in multiple planes.

9. The securement devices of claim 8 where the base pad further comprising a solid yet generally flexible material such as but not limited to fabric, foam, or other synthetic materials.

10. The securement device of claim 8 where the base comprising the use of any medical-grade adhesive safe for use on skin and further comprising at least one set of release paper attached to the bottom surface of the base as a means of temporarily covering the adhesive until the device may be optimally positioned.

11. The securement device of claim 8 where the platform is generally rectangular and where the angle of decline about the platform is between 10 and 45 degrees.

12. The securement device of claim 8 where the platform, and securement member are comprising a solid material such as but not limited to any injection-moldable plastics.

13. The securement device of claim 8 where the securement member having a lengthwise slit of predetermined size running alone one side as a means of entrapping the upper edge of the retractable wall as to hold it in a closed position.

14. The securement device of claim 8 where the means of connection between the platform and the retractable wall, and the means of connection between the platform and the securement member comprising but not limited to any living hinge or other mechanism allowing flexion.

15. A securement device for IV devices and catheters comprising:
a base pad having an upper side and an opposite underside where at least a portion of the undersurface having an adhesive thereon for securing the device to the body;
a securement member attached to the upper side of the base pad having a first and opposite second side extending up from a common base having a central channel of predetermined width within where an IV device will securely rest therein, and attaching to at least one side of the securement member, a latching device providing a means of securing and holding medical tubing;
a platform attached to the upper side of the base pad and extending from the securement member having an angle of decline about its upper surface providing a means of maintaining an angle of IV device insertion and a means of protection between the IV device and the body;
whereby the combined structure of the base pad, the platform, and the base, sides, and latching device of the securement member provide for securing IV devices to the body and additionally providing direct support and stabilization to the IV devices and tubing while also mitigating movement and rotation of the IV device in multiple planes.

16. The securement device of claim 15 where the base pad further comprising a solid yet generally flexible material such as abut not limited to fabric, foam, or other synthetic materials.

17. The securement device of claim 15 where the base pad has a plurality of extensions at least two of which extend toward the point where an IV device enters the skin.

18. The securement device of claim 15 where the angle of decline about the platform is between 10 and 45 degrees.

19. The securement device of claim 15 where the securement member and latching device are comprising a solid material such as but not limited to any injection-moldable plastics.

20. The securement device of claim 15 where the base pad further comprised the use of any medical-grade adhesive safe for use on skin, and further comprising at least one set of release paper attached to the bottom surface of the base as a means of temporarily covering the adhesive until the device may be optimally positioned.
